(19) United States
(12) Patent Application Publication
(10) Pub. No.: US 2014/0170090 A1
(43) Pub. Date: Jun. 19, 2014

(54) REDUCED-ALCOHOL SUNSCREEN COMPOSITIONS AND METHODS

(71) Applicant: Holly E. THAGGARD, San Antonio, TX (US)

(72) Inventor: Holly E. THAGGARD, San Antonio, TX (US)

(73) Assignee: Taylor James, LLC, San Antonio, TX (US)

(21) Appl. No.: 14/107,703

(22) Filed: Dec. 16, 2013

Related U.S. Application Data
(60) Provisional application No. 61/738,269, filed on Dec. 17, 2012.

(51) Int. Cl. A61K 8/81; A61K 8/35; A61K 8/97; A61K 8/67; A61K 8/37; A61K 8/40; A61Q 17/04; A61K 8/34

(52) U.S. Cl. CPC A61K 8/8152; A61Q 17/04; A61K 8/35; A61K 8/34; A61K 8/676; A61K 8/37; A61K 8/40; A61K 8/97
USPC 424/59

(57) ABSTRACT

Sunscreen compositions including a sunscreen component including avobenzone in an amount sufficient to provide at least UVA protection, an alcohol component present in a reduced-amount, and an antioxidant component present in an amount sufficient to impart an antioxidant effect to the sunscreen composition. In one aspect, the composition further includes a water-penetrating polymer in an amount sufficient to facilitate application of the composition in the presence of water. Methods of preparing and applying such reduced-alcohol compositions are also included.

### CROSS-REFERENCE TO RELATED APPLICATION

This application claims the benefit of and priority to U.S. Provisional Application No. 61/738,269, filed Dec. 17, 2012, the contents of which is hereby incorporated herein in its entirety by express reference thereto.

### TECHNICAL FIELD

The invention is directed to sunscreen compositions that include: a sunscreen component including avobenzone in an amount sufficient to provide at least UVA protection; an alcohol component present in an amount of about 20% to no more than 65% of the total sunscreen composition; and an antioxidant component present in an amount sufficient to impart an antioxidant effect to the sunscreen composition. The compositions preferably include a water-penetrating polymer component to facilitate application of the composition in the presence of water. The invention further relates to methods of preparing and applying such compositions to the skin, including wet skin.

### BACKGROUND

The undesirable effects of overexposure to sunlight are well known, and can result in not only uncomfortable sunburn but in premature aging of skin, wrinkles, loss of skin elasticity, dermatosis, and skin cancer. Sunscreen is often desired to protect the skin from these, and other, adverse effects of solar radiation. The most dangerous solar radiation tends to be ultraviolet (UV) radiation, such as at wave lengths lower than 400 nm which includes both UV-A and UV-B radiation. In general, the UV-A light refers to ultraviolet light having a wave length of 320 to 400 nm and UV-B light refers to ultraviolet light have a wave length of 280 to 320 nm.

Conventional sunscreen compositions are typically in the form of a liquid, either a lotion or a cream. These compositions are either oil or water-based. The water-based emulsion forms serve mainly as an aid to disperse the active ingredients topically. The carrier water evaporates and leaves a thin film of active ingredients plus excipients deposited on the skin. The remaining film contains the product that protects the skin from ultraviolet radiation. Sunscreen compositions are typically rated by their sun protection factor (SPF), a measure of the protection from the sun afforded by the sunscreen composition. Compositions having higher SPF values provide more protection from solar radiation, but these are also known to wear off particularly when exposed to wet conditions.

UV-A absorbers have a tendency to degrade when exposed to light. For example, the UV-A absorber avobenzone (commercially available, for example, as PARSOL 1789, EUSOLEX 9020, ESCALOL 517, and others; INCI Butyl Methoxydibenzoylmethane) is known to degrade when exposed to light. See, e.g., http://en.wikipedia.org/wiki/Avobenzone. The photo stability of avobenzone remains highly formulation dependent and therefore unpredictable generally. See htp://www.cosmeticsdatabase.com/special/sunscreens/summary.php.

In addition to the known instability of UV-A absorbers, spray sunscreen compositions are known in the art but typically include other absorbing components like oxybenzone that may have deleterious health effects, and often contain significant amounts of alcohol of 70% or more, typically to enable drying of the applied sunscreen before it is washed off such as from sweat or other water sources. Alcohol dries far more quickly than water, and by using alcohol in place of water in sunscreen compositions they can dry more quickly. Alcohol, however, is highly flammable and can also be damaging to skin because of its rapid drying effect.

Thus, there is a need in the art for reduced-alcohol sunscreen compositions that still dry sufficiently rapidly, while still providing a healthy sunscreen absorber to minimize or reduce the skin damage from the sun.

### SUMMARY

The invention as described in the present disclosure encompasses sunscreen compositions including a sunscreen component including avobenzone in an amount sufficient to provide at least UVA protection, an alcohol component present in an amount of about 20% to no more than 65% of the total sunscreen composition, and an antioxidant component present in an amount sufficient to impart an antioxidant effect to the sunscreen composition. In a preferred embodiment, the composition further includes a water-penetrating polymer in an amount sufficient to facilitate application of the composition in the presence of water. In a more preferred embodiment, the water-penetrating polymer includes an alkyl maleate/acrylate copolymer.

In another aspect, the invention encompasses a sunscreen composition that includes a sunscreen component including avobenzone, homosalate, octinoxate, octisalate, and octocrylene each present in an amount sufficient to collectively provide UVA and UVB protection, an alcohol component present in an amount of about 20 weight percent to no more than 65 weight percent of the total sunscreen composition, a water-penetrating polymer comprising an alkyl maleate/acrylate copolymer in an amount sufficient to repel water, and an antioxidant component including at least one vitamin present in an amount sufficient to impart an antioxidant effect to the sunscreen composition.

In one embodiment, the water-penetrating polymer is present in an amount of about 0.1 to about 5 weight percent of the total composition. In a preferred embodiment, the water-penetrating polymer is formed from a butyl maleate/isobornyl acrylate-type copolymer. In another embodiment, the sunscreen composition is at least substantially free of oxybenzone, at least substantially free of Vitamin A, or both. In a preferred embodiment, the sunscreen composition is entirely free of oxybenzone and Vitamin A.

In one embodiment, the sufficient amount of sunscreen component includes 2.2 weight percent to about 7.5 weight percent avobenzone. In a preferred embodiment, the sufficient amount of sunscreen component includes about 2.5 weight percent to about 4.5 weight percent avobenzone. In another embodiment, the alcohol component is present in an amount of about 30 weight percent to about 60 weight percent of the total composition. In a preferred embodiment, the alcohol component is present in an amount of about 40 weight percent to about 55 weight percent of the total composition. In one embodiment, the sunscreen composition preferably including the water-penetrating polymer (or hydrophobic polymer) imparts sufficient water penetration to provide UVA and UVB sunscreen efficacy for at least 80 minutes.

In another embodiment, the sufficient amount of the antioxidant component is from about 0.001 to about 1 weight percent of the total composition. In a further embodiment, the antioxidant component includes at least one vitamin and at least one organic extract. In a preferred embodiment, the antioxidant component includes Vitamin C in oil soluble form, acai extract, and green tea extract. In a preferred embodiment, the sunscreen component, alcohol component, antioxidant component, and water-penetrating polymer are collectively present in relative amounts to provide a clear sunscreen composition.

In another aspect, the disclosure encompasses a sunscreen composition includes a sunscreen component that includes avobenzone, homosalate, octinoxate, octisalate, and octocrylene each present in an amount sufficient to collectively provide UVA and UVB protection; an alcohol component present in an amount of about 20% to no more than 65% of the total sunscreen composition; a water-penetrating polymer including an alkyl maleate/acrylate copolymer in an amount sufficient to repel water; and an antioxidant component including at least one vitamin present in an amount sufficient to impart an antioxidant effect to the sunscreen composition.

In a further aspect, the disclosure encompasses a method of preparing a continuous-spray sunscreen composition, which includes providing a sunscreen component including avobenzone in an amount sufficient to provide UVA protection; providing an alcohol component present in an amount of about 20% to 65% of the total sunscreen composition; providing an antioxidant component present in an amount sufficient to impart an antioxidant effect to the sunscreen composition; providing a hydrophobic polymer present in an amount sufficient to repel water contacting the composition; and combining the sunscreen component, the alcohol component, the antioxidant component, and the hydrophobic polymer so as to form a continuous-spray sunscreen. In a preferred embodiment, the sunscreen component of the continuous-spray sunscreen composition further includes at least three of homosalate, octinoxate, octisalate, or octocrylene, while a more preferred embodiment, the sunscreen component includes all four of homosalate, octinoxate, octisalate, and octocrylene.

In one embodiment, the sunscreen composition is selected to be at least substantially free of oxybenzone. In another embodiment, the sufficient amount of sunscreen component includes 2.2 weight percent to about 7.5 weight percent avobenzone. In yet another embodiment, the alcohol component is provided in an amount of about 30 weight percent to about 60 weight percent of the total composition. In yet another embodiment, the antioxidant component includes at least one vitamin and at least one extract and is provided in an amount of about 0.001 to about 1 weight percent of the total composition.

In another aspect, the disclosure encompasses a method for applying a reduced-alcohol sunscreen composition to skin, which includes providing the reduced-alcohol sunscreen composition of claim 2 in a pressurized container adapted to direct a stream of the sunscreen composition in liquid form from the container in a selected direction, and activating a release mechanism to direct the liquid sunscreen composition in the selected direction onto a person's skin. In one embodiment, the skin is wet and the sunscreen composition imparts UVA and UVB sunscreen efficacy with an SPF of at least about 20, preferably at least about 30, for at least 80 minutes to the wet skin. In another embodiment, the sunscreen component, alcohol component, antioxidant component, and hydrophobic polymer are collectively present in relative amounts in the reduced-alcohol sunscreen composition to provide a clear sunscreen composition as applied to the skin. In yet a further embodiment, the stream is a spray or mist and the release mechanism is triggered by pressure applied to the pressurized container to direct the sunscreen composition on the person's skin.

Various of these embodiments and those further discussed herein should be understood to be applicable to various aspects of the disclosure even if described only in connection with one particular aspect.

### BRIEF DESCRIPTION OF THE DRAWINGS

The present disclosure is best understood from the following detailed description when read with the accompanying FIGURE(s). It is emphasized that, in accordance with the standard practice in the industry, various features are not drawn to scale. In fact, the dimensions of the various features may be arbitrarily increased or reduced for clarity of discussion.

FIG. 1 is an illustration of a graph showing mean absorbance and critical wavelength as a measure of wavelength (nm) according to one or more aspects of the present disclosure.

### DETAILED DESCRIPTION OF THE PREFERRED EMBODIMENTS

The photoprotectant cosmetic compositions of the invention as disclosed herein include a sunscreen composition that includes a sunscreen component including avobenzone in an amount sufficient to provide at least UVA protection, an alcohol component in a reduced amount compared to conventional spray sunscreen formulations, and an antioxidant component present in an amount sufficient to impart an antioxidant effect to the sunscreen composition. Preferably, the composition also includes a water-penetrating polymer in an amount sufficient to facilitate application of the composition in the presence of water. The alcohol component is present in an amount of about 20 weight percent to no more than 65 weight percent of the total sunscreen composition. Preferably, the alcohol present in the composition as a whole is at least about 30% less than conventional spray-on sunscreen compositions. These compositions are preferably formulated as a liquid, typically a solution or suspension of suitable viscosity to be directed as a stream out of a container, such as in spray or mist form, to be applied to a user's skin and retained thereon. The invention further relates to methods of photoprotecting the skin of a subject from the damaging effects of UV radiation by applying the sunscreen compositions herein.

The sufficient amount of sunscreen component preferably includes at least 2.2 weight percent to about 7.5 weight percent avobenzone, more preferably about 2.5 weight percent to about 5 weight percent avobenzone. In one preferred embodiment, it refers to avobenzone present in amounts of about 2.8 weight percent to 4.5 weight percent, and in an exemplary embodiment it refers to 3 weight percent of the total composition. The sunscreen component in preferred embodiments includes at least two, preferably at least three, and more preferably at least four of homosalate, octinoxate, octisalate, and octocrylene. Homosalate is also known as 3,3,5-Trimethylcyclohexyl 2-hydroxybenzoate. Octinoxate is chemically known as octyl methoxycinnamate. Octisalate, also known as 2-ethylhexyl salicylate, helps provide protection against UV-B energy. Octocrylene is also known as 2-ethylhexyl 2-cyano-3,3-diphenyl-2-propenoate. When present, each of these sunscreen components is included in an amount of about 2.5 weight percent to 20 weight percent when only two of these are included with avobenzone, preferably about 3 weight percent to about 15 weight percent such as when three are included with avobenzone, and more preferably about 3 weight percent to about 12.5 weight percent when all four are included with avobenzone. In exemplary embodiments, the homosalate and octocrylene are each present in an amount of about 7.5 weight percent to about 12.5 weight percent; the octinoxate is present in an amount of about 5 weight percent to about 10 weight percent; the octisalate is present in an amount of about 2.5 weight percent to about 7.5 weight percent; each based on total composition weight. Thus, in a preferred embodiment, the sunscreen composition includes a sunscreen component present in an amount of about 12.2 weight percent to about 57.5 weight percent, with other preferred total weights being calculated based on the preferred and exemplary amounts set forth herein.

The alcohol component is preferably present in an amount of about 30 weight percent to about 60 weight percent, more prefer in an amount of about 40 weight percent to about 55 weight percent, of the total composition. An exemplary alcohol content is about 32 weight percent to about 50 weight percent of the total composition, while another is about 35 weight percent to about 48 weight percent of the total composition. One or more alcohols may be used, and they are selected based on the guidance herein so as to facilitate drying of the applied sunscreen compositions. The alcohols may be organic, inorganic, or a mixture thereof.

The water-penetrating polymer is preferably a hydrophobic polymer, such as one or more alkyl maleate/acrylate copolymers. Other suitable hydrophobic polymers safe for topical administration may be envisioned, as well. Preferably, the alkyl is a lower alkyl maleate, and more preferably a C₂₋₈ maleate. In an exemplary embodiment, the alkyl maleate is butyl maleate. While various acrylate materials may be selected, the acrylate in an exemplary embodiment is isobornyl acrylate. The water-penetrating polymer is preferably included and when present is included in an amount of about 0.1 to about 5 weight percent, preferably about 0.2 to about 2.5 weight percent, and more preferably about 0.5 to 1.5 weight percent of the total composition.

Because of potentially deleterious health effects, particularly on the skin, the sunscreen compositions preferably are at least substantially free of oxybenzone, Vitamin A, or both. More preferably in some embodiments, the sunscreen composition is entirely free of oxybenzone, Vitamin A, or both. By “substantially free” is meant less than about 2 weight percent, preferably at least about 1 weight percent, and more preferably at least about 0.1 weight percent, of the excluded component.

The sunscreen compositions impart sufficient water penetration to be applied to the skin even when wet, and repel water sufficiently to provide UVA and UVB sunscreen efficacy for at least 80 minutes even in wet conditions (including sweat). Without being bound by theory, it is believed that water-penetrating polymer provides the bulk of this efficacy.

The antioxidant component may be included in amounts from about 0.003 to about 1.5 weight percent of the total composition. Preferably, the antioxidant component may be included in an amount of about 0.01 to about 0.75 weight percent, and more preferably about 0.5 to about 0.5 weight percent, of the total composition. In an exemplary embodiment, the antioxidant component is present in an amount of about 0.21 weight percent of the composition. The antioxidant component comprises at least one vitamin and at least one organic extract, preferably vitamin C, and either acai extract or green tea extract, or both. These extracts may be obtained, for example, from euterpe oleracea fruit and camellia sinensis leaf, respectively. The vitamin C is preferably in an oil soluble form. An exemplary oil soluble vitamin C is tetrahexyldecyl ascorbate. The vitamin C component is preferably present in an amount of about 0.001 weight percent to about 0.5 weight percent, preferably about 0.005 weight percent to about 0.1 weight percent, and more preferably about 0.008 weight percent to about 0.05 weight percent, of the total composition. In an exemplary embodiment, the vitamin C component is present in an amount of about 0.01 weight percent of the composition. The organic extract portion is preferably present in an amount of about 0.001 weight percent to 1 weight percent, preferably about 0.01 weight percent to about 0.5 weight percent, and more preferably about 0.05 weight percent to about 0.25 weight percent, of the total composition. In an exemplary embodiment, the extract portion of the antioxidant is present in an amount of about 0.2 weight percent of the total composition.

The sunscreen component, alcohol component, antioxidant component, and water-penetrating polymer are collectively present in the sunscreen compositions in relative amounts to provide a clear sunscreen composition, as distinguished from an opaque or cloudy formulation as is the case with many cream, milk, and lotion-type sunscreens.

In one exemplary embodiment, the sunscreen composition includes a sunscreen component that includes avobenzone, homosalate, octinoxate, octisalate, and octocrylene each present in an amount sufficient to collectively provide UVA and UVB protection, an alcohol component present in an amount of about 20 weight percent to no more than 65 weight percent, preferably about 30 weight percent to about 55 weight percent, of the total sunscreen composition, a water-penetrating polymer including an alkyl maleate/acrylate copolymer in an amount sufficient to repel water, and an antioxidant component comprising at least one vitamin present in an amount sufficient to impart an antioxidant effect to the sunscreen composition.

The term sunscreen, as used herein, is a composition that when applied to the skin forms a coating, or film, where applied that protects the skin against the damaging effects of ultraviolet (UV) exposure. The effectiveness of a sun screen, i.e., its ability to block UV radiation is typically expressed as a sun protection factor or “SPF” rating. Sunscreens protect the skin by absorbing UV radiation before it can interact with and damage the skin, however, their efficacy is often limited in time. Formulation of the sunscreen can affect the length of time a photoprotectant effect is provided, particularly in view of the known instability and unpredictability of avobenzone formulations.

The photoprotectant cosmetic compositions typically have an SPF rating of at least 10 to about 65, preferably at least about 15. In one embodiment, the SPF rating is above about 20. In one embodiment, the SPF rating is above about 22. In one embodiment, the SPF rating is at least about 30. In one embodiment, the SPF rating is at least about 35. In one embodiment, the SPF rating is at least about 40. In one embodiment, the SPF rating is at least about 45. A preferred SPF rating of the composition is from about 20 to about 62, and another preferred SPF rating is from about 28 to about 60. In a preferred embodiment, the SPF is about 50 to about 60. In each case, the above SPF ratings are for at least about 80 minutes. SPF rating is determined using FDA approved method. See U.S. Food and Drug Administration. Labeling and Effectiveness Testing; Sunscreen Drug Products for Over-the-Counter Human Use; Final Rule; 21 CFR Parts 201 and 310. Federal Register, Vol. 76, No. 117, Jun. 17, 2011. pp. 35660-35665 (“FDA Final Rule”).

The compositions herein can include a variety of other components known to be included in skin care compositions. The CTFA Cosmetic Ingredient Handbook, Seventh Edition, 1997 and the Eighth Edition, 2000, which is incorporated herein by reference in its entirety, describes a wide variety of cosmetic and pharmaceutical ingredients commonly used in skin care compositions, which are suitable for use in the compositions of the present invention. Illustrative examples of the functional classes of these ingredients include, but are not limited to, one or more of the following: absorbents, abrasives, anticaking agents, antifoaming agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticizers, reducing agents, skin bleaching agents, skin-conditioning agents (emollient, humectants, miscellaneous, and occlusive), skin protectants, solvents, foam boosters, hydrotropes, solubilizing agents, suspending agents (nonsurfactant), sunscreen agents, ultraviolet light absorbers, SPF boosters, waterproofing agents, and viscosity increasing agents (aqueous and nonaqueous).

In an exemplary embodiment, the sunscreen compositions may additionally preferably include an oil component. The oil component preferably includes one or more of the following (or an extract thereof as noted): Cananga Odorata Flower Oil, Citrus Aurantifolia (Lime) Oil, Citrus Grandis (Grapefruit) Peel Oil, Citrus Limon Fruit Oil, Citrus Nobilis (Mandarin Orange) Peel Oil, Citrus Reticulata (Tangerine) Peel Oil, Citrus Sinensis Peel Oil, Helianthus Annuus (Sunflower) Seed Oil, Lavandula Angustifolia (Lavender) Oil, Mentha Piperita (Peppermint) Oil, Mentha Viridis (Spearmint) Leaf Oil, and Vitis Vinifera (Grape) Seed Oil, and any combination thereof. Preferably, two or more, more preferably three or more, and even more preferably four or more of these oils are included in the oil component. In one preferred embodiment, the oil component includes all but two, preferably all but one, and most preferably all of these listed oils. The oil may be present in any detectable amount, and may be used to help complete the remainder of the composition after the sunscreen component, antioxidant component, alcohol, and water-penetrating polymer component are accounted.

In another embodiment, one or more additional preferred materials included in the sunscreen compositions include: Caprylic/Capric Triglyceride, Dicaprylyl Carbonate, Diisopropyl Adipate, Isodecyl Neopentanoate, Tridecyl Neopentanoate, or a combination thereof. These additional preferred materials may be included in sufficient amounts to facilitate the water-penetrating polymer component's ability to penetrate through water for application on wet skin, to repulse water to resist being washed off by water, or any other desired purpose in amounts that, with the oil component and other components disclosed, may preferably account for the remainder of the compositions disclosed herein.

The compositions of the invention are prepared by admixing the components using procedures well known to formulators of sunscreen and other chemical compositions, particularly based on the guidance provided herein. Preferably, the composition is formulated as a liquid, preferably one adapted to spray or mist out of a container onto skin for application. The sunscreen compositions herein are preferably packaged as a spray or mist, and may be provided in a pressurized container to facilitate administration. The spray or mist is preferably continuous-spray or continuous-mist, to facilitate application of the sunscreen composition across various portions of a user's skin.

One method of preparing a continuous-spray sunscreen composition includes providing a sunscreen component comprising avobenzone in an amount sufficient to provide UVA protection, providing an alcohol component present in an amount of about 20 weight percent to 65 weight percent, of the total sunscreen composition, providing an antioxidant component present in an amount sufficient to impart an antioxidant effect to the sunscreen composition, providing a hydrophobic polymer present in an amount sufficient to repel water contacting the composition, and combining the sunscreen component, the alcohol component, the antioxidant component, and the hydrophobic polymer so as to form a continuous-spray sunscreen. Any of the compositions described herein may be used in any such methods herein.

The reduced-alcohol sunscreen compositions disclosed herein may also be applied to skin by providing a reduced-alcohol sunscreen composition disclosed herein in a pressurized container adapted to direct a stream of the sunscreen composition in liquid form from the container in a selected direction, and activating a release mechanism to direct the liquid sunscreen composition in the selected direction onto a person's skin. In many cases, the user's skin is wet (e.g., from sweat or swimming) yet the sunscreen compositions described herein still imparts UVA and UVB sunscreen efficacy with an SPF of at least about 20 for at least 80 minutes to the wet skin. This can be achieved using less alcohol than conventional sunscreen spray-on products, and with a preferred sunscreen component including avobenzone, preferably in the absence of oxybenzone. The directed stream of sunscreen composition preferably includes a spray or mist, and the release mechanism is triggered by pressure applied to the pressurized container to direct the sunscreen composition on the person's skin.

### EXAMPLES

The following example is set forth to assist in understanding the invention and should not be construed as specifically limiting the invention described and claimed herein.

### Example 1

### Preparation of a Composition According to One Embodiment

A composition was prepared containing the following components with listed percentages based on total composition weight: Active Ingredients: Avobenzone (3.0%), Homosalate (10.0%), Octinoxate (7.5%), Octisalate (5.0%), and Octocrylene (10.0%). Inactive Ingredients: Alcohol Denat., Camellia Sinensis Leaf Extract, Cananga Odorata Flower Oil, Caprylic/Capric Triglyceride, Citrus Aurantifolia (Lime) Oil, Citrus Grandis (Grapefruit) Peel Oil, Citrus Limon Fruit Oil, Citrus Nobilis (Mandarin Orange) Peel Oil, Citrus Reticulata (Tangerine) Peel Oil, Citrus Sinensis Peel Oil, Dicaprylyl Carbonate, Diisopropyl Adipate, Euterpe Oleracea Fruit Extract, Helianthus Annuus (Sunflower) Seed Oil, Isodecyl Neopentanoate, Lavandula Angustifolia (Lavender) Oil, Mentha Piperita (Peppermint) Oil, Mentha Viridis (Spearmint) Leaf Oil, Tetrahexyldecyl Ascorbate (Vitamin C), Tridecyl Neopentanoate, VA/Butyl Maleate/Isobornyl Acrylate Copolymer, and Vitis Vinifera (Grape) Seed Oil.

### Example 2

### Testing for “Broad Spectrum” Qualification of the Composition of Example 1

A test sample labeled as SPF 50 sunscreen spray was received and assigned AMA Lab No.: M-7372.

Study Objectives:

The sample (AMA Lab No.: M-7372; Client No.: Supergoop SPF 50 Sunscreen Spray; Sample ID: 06-JD-115D) was evaluated according to the broad spectrum testing method (21 CFR 201.327.(j)) as defined by the Final Monograph; “Labeling and Effectiveness testing; Sunscreen Drug Products for Over-the-Counter Human Use”, Final Rule, 21 CFR Parts 201 and 310, (FR Doc. 2011-14766 Filed Jun. 16, 2011 at 8:45 am; Publication Date: Jun. 17, 2011, Docket No. FDA-1978-N-0018, RIN 0910-AF43) using Labsphere's UV-20005 Benchtop Sunscreen Analyzer. The Solar Light Xenon Arc Fade Test UV Simulator—Model 16S-300-003 V4.0 or LS1000-6S-UV was used as UV source of pre-irradiation.

Plate (Substrate):

PMMA Plates Sa: 6 micrometers (Sa requirement: 2 to 7 micrometers)

Application Area: 5 cm×5 cm=25 cm² (Area requirement: min. 16 cm²)

Manufacturer: HeliosScreen Laboratoire

Designation: HD6 2009 000109

Methodology:

Quantity Applied:

Sunscreen product was applied to the roughened PMMA plate (roughened side uppermost) by weight, at an application rate of 0.75 mg/cm² using a positive-displacement automatic pipette.

Spreading Technique:

The type of spreading action to be employed when applying the test product consists of two phases. Phase 1: Spreading with a very light pressure for approximately 30 seconds. Phase 2: Spreading with greater pressure for approximately 30 seconds. The treated sample is then allowed to equilibrate for 15 minutes in the dark at ambient temperature to help facilitate formation of a standard stabilized product film.

Pre-Irradiation UV Dose (PIP):

To account for lack of photostability, the test product is applied on the PMMA plate and irradiated with a fixed dose of UV radiation. The pre-irradiation dose to be delivered is calculated as follows:

Dose=4 MED=4×200 J/m²-eff(800 J/m²-eff)

Where: MED—Minimal Erythemal Dose, the lowest UV dose that produces skin reddening.

1 MED=200 J/m²-eff

UV Source (Solar Simulator) Emission Spectrum:

Solar simulator is filtered so that it provides a continuous emission spectrum from 290 to 400 nanometers (nm) with a limit of 1,500 watts per square meter (W/m²) on total solar simulator irradiance for all wavelengths between 250 and 1400 nm and the following percentage of erythema-effective radiation in each specified range of wavelengths:

| Wavelength range (nm) | Erythemal Contribution (%) |
|---|---|
| <290 | <0.1 |
| 290-310 | 46.0-67.0 |
| 290-320 | 80.0-91.0 |
| 290-330 | 86.5-95.0 |
| 290-340 | 90.5-97.0 |
| 290-350 | 93.5-98.5 |
| 290-400 | 93.5-100.0 |

In addition, UVA II (320-340 nm) irradiance is >20% of the total UV (290-400 nm) irradiance. UVA I (340-400 nm) irradiance is >60% of the total UV irradiance.

The emission spectrum of the solar simulator was determined using a radiometer with a response weighted to match the spectrum in ISO 17166 CIE S 007/E entitled “Erythemal reference action spectrum and standard erythema dose,” dated 1999 (First edition, 1999 Dec. 15; corrected and reprinted 2000 Nov. 15), which is incorporated by reference in accordance with 5 U.S.C. §552(a) and 1 C.F.R. part 51.

Transmittance Measurements:

The transmittance values are measured at 1 nanometer intervals on three different plates with a minimum of 5 measurements per plate. Measurements of spectral irradiance transmitted for each wavelength λ through control PMMA plates coated with 15 microliters of glycerin (no sunscreen product) are obtained from 5 different locations on the PMMA plate [C1(λ), C2(λ), C3(λ), C4(λ), and C5(λ)]. In addition, a minimum of 5 measurements of spectral irradiance transmitted for each wavelength A. through the PMMA plate covered with the sunscreen product are similarly obtained after pre-irradiation of the sunscreen product [P1(λ), PC2(λ), P3(λ), P4(λ), and P5(λ)]. The mean transmittance for each wavelength, T̅(̅λ̅)̅, is the ratio of the mean of the C(λ) values to the mean of the P(λ) values, as follows:

T̅(̅λ̅)̅: Σⁿ₁P(λ)/n

Σⁿ₁C(λ)/n
Where: n≧5
   Calculation of mean absorbance values:

Mean transmittance values, T(λ), are converted into mean absorbance values, A̅(̅λ̅)̅, at each wavelength by taking the negative logarithm of the mean transmittance value as follows:

=−log T(λ)

Determination of Critical Wavelength:

Critical wavelength measurements are used to measure the breadth of the UV absorbance curve. Critical wavelength (λc) is the wavelength at which the area under the absorbance curve represents 90 percent of the total area under the curve in the UV region. This is expressed mathematically as:

∫₂₉₀^{λc} A̅(̅λ̅)̅dλ=0.9∫₂₉₀⁴⁰⁰ A̅(̅λ̅)̅dλ

Where: λ_{c}—Critical wavelength
A(λ)—Mean absorbance at each wavelength
dλ—Wavelength interval between measurements
A mean critical wavelength of λ_{c}=370 nm or greater is classified as broad spectrum protection.

### Results:

Critical Wavelength: (requirement: minimum λc=370 nm)

Critical Wavelength Values After Pre-lrradiation Procedure
UV Source Irradiance Output: 6.50 MED/h
Iradiation Time (Single Plate): 2215 sec

| | Location 1 | Location 2 | Location 3 | Location 4 | Location 5 |
|---|---|---|---|---|---|
| Plate 1 | 375 | 375 | 375 | 375 | 375 |
| Plate 2 | 375 | 375 | 375 | 375 | 375 |
| Plate 3 | 375 | 375 | 375 | 375 | 375 |

| | |
|---|---|
| Average: | 375.00 nm |

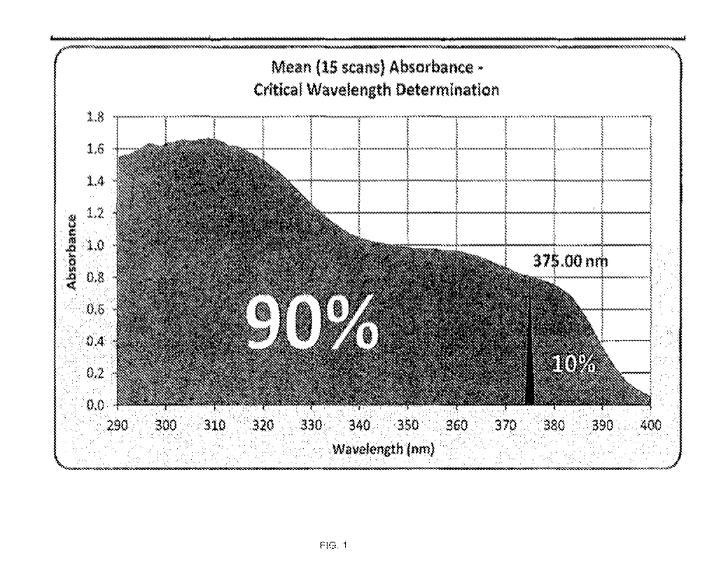

The Critical Wavelength of the above test material (AMA Lab No.: M-7372; Client No.: Supergoop SPF 50 Sunscreen Spray; Sample ID: 06-JD-115D) is 375.00 nm as shown in FIG. 1, and satisfies the criteria for “Broad Spectrum” labeling (minimum of 370 nm required).

### Example 3

### SPF Testing (80 Minute Water Resistant) of the Composition of Example 1

Objective:

A panel was convened to evaluate the effectiveness of a test material as a sunscreen product by determining the Sun Protection Factor (SPF) on human skin as defined by the FDA Sunscreen Final Rule; 21 CFR Parts 201 and 310 [Docket No. FDA-1978-N-0018](formerly Docket No. 1978N-0038), RIN 0910-AF43, Labeling and Effectiveness Testing; Sunscreen Drug Products For Over-the Counter Human Use [FR Doc. 2011-14766 Filed Jun. 16, 2011; Publication Date: Jun. 17, 2011] using Xenon arc solar simulator as the UV source. This test was conducted prior to and immediately following a 80 minute water immersion experiment which was carried out under controlled conditions as described in the above mentioned FDA Sunscreen Final Rule and the Procedures below.

### Panel Composition:

Healthy volunteers over the age of 18 years were recruited for this study. The panel consisted of fair-skin individuals with Fitzpatrick Skin Types I, II or III defined as follows (Federal Register Vol. 64, No. 98: 27690, 1999):

Type I—Always burns easily; never tans*

Type II—Always burns easily; tans minimally*

Type III—Burns moderately; tans gradually*

* Based on the first 30 to 45 minutes sun exposure after a winter season of no sun exposure.

Artificial Light Source:

The light source employed was a 150 watt Xenon Arc Solar Simulator (Solar Light Co., Philadelphia, Pa., Model 14S, Model 15S or Model 16S) having a continuous emission spectrum in the UVB range from 290 to 400 nm. Xenon arc was selected on the basis of its black body radiation temperature of 6000° K which produces continuous UV spectra (all wavelengths) substantially equivalent to that of natural sunlight (Berger, D.S., “Specification and Design of Solar Ultraviolet Simulators,” J. Invest. Dermatol. 53: 192-199, 1969).

This device was equipped with a dichroic mirror (which reflects all radiation below 400 nm) and works in conjunction with a 1 mm thick Schott WG-320 filter (which absorbs all radiation below 290 nm) to produce simulation of the solar UVA-UVB spectrum. A 1 mm thick UG 11 filter (black lens) was added to remove reflected (infra-red, greater than 700 nm) heat and remained visible radiation. UVB radiation was monitored continuously during exposure using a Model DCS-1 Sunburn UV Meter/Dose Controller System (Solar Light Co.) formerly known as the Robertson-Berger Sunburn Meter (R-B meter). Measurements were taken at a position within 8 mm from the surface of the skin. The size of the exposure site is £ 1 cm². The solar simulator was allowed a warm up time of at least 15 minutes before use and power supply output was recorded.

Procedure:

(A) Static SPF Determination (Including 7% Padimate Q/3% Oxybenzone Standard)

The infrascapular area of the back to the right and left side of the midline was used. Within this area, 30 cm² rectangular test sites were delineated with a gentian violet surgical skin marker. Sites were observed to ensure uniform pigmentation, skin tone and texture, and absence of warts, moles, nevi, scars, blemishes and active dermal lesions. Any areas that might be expected to produce erratic results were not used for UV exposures.

The procedure for this study is outlined in the Federal Register/Vol. 76, No. 117, 21 CFR Parts 201 and 310 published on Friday Jun. 17, 2011. One test site area served to determine each subject's Minimal Erythema Dose (MED). A minimum of five UV exposures was administered within this site. The individual subject's MED is the shortest time of exposure that produces minimally perceptible erythema at 16 to 24 hours post irradiation.

The test material and 7% Padimate 0/3% Oxybenzone standard were shaken and/or swirled with a glass rod before use and were evenly applied using plastic volumetric syringes to rectangular areas measuring ‘3 cm×10 cm (30 cm²) for a final concentration of 2.0 mg/cm². Evenness of application was verified by observation with a Wood's Lamp. An adjacent test site was then selected to perform a static determination on the test substance, as above, prior to the immersion test.

Fifteen minutes after application, a protected site received a series of five UV exposures based upon previously determined MED. All immediate responses were recorded after UV radiation exposure from the solar simulator. The UV exposures for 7% Padimate 0/3% Oxybenzone, in-house water resistant control and test material were calculated from previously determined MED and the intended SPF as follows:

SPF 16.3: MED times 0.76x, 0.87x, 1.00x, 1.15x and 1.32x

SPF 15: MED times 0.69x, 0.83x, 1.00x, 1.20x and 1.44x

SPF 50: MED times 0.76x, 0.87x, 1.00x, 1.15x, and 1.32x where x equals the expected SPF of the product.

(B) 80 Minute Water Resistant Determination (Including In-House Water Resistant Control)

This test was employed to determine the substantivity of a test product and its ability to resist water immersion. On the day of the test, following exposure of the 7% Padimate 0/3% Oxybenzone standard, MED's and static determination, another test site was designated. One other adjacent site was selected to perform a water resistant determination of an in-house 80 minute water resistant standard with a known SPF as a control.

The water resistant SPF value was determined by the product's ability to resist an 80-minute period of water immersion, achieved through the following test regimen: After application of the sunscreen product followed by the waiting period, a total of 80 minutes water immersion was scheduled; 20 minute intervals in the water, followed by 15 minute rest intervals (without towel drying). Immersion was achieved indoors in a circulating whirlpool maintained at 23 to 32° C. for these testing procedures. The pool and air temperature and the relative humidity were recorded. At the conclusion of the water immersion/rest period cycle, the test sites were allowed to air-dry without toweling prior to exposure from the solar simulator, as described.

Evaluation of Responses:

Sixteen to twenty-four hours post exposure, the subjects were instructed to return to the testing facility for evaluation of delayed erythemic responses. The technician who evaluated the MED did not know the identity of the test product application sites and UV exposures. Also he/she was not the same person to have applied the sunscreen product to the test site or administered the doses of UV radiation.

Protected MED

Final unprotected MED

Visual grading scale:

0=No Erythema

?=Questionable Erythema

1=Minimal Erythema

2=Slight Erythema

3=Well-Defined Erythema

4=Erythema and Edema

5=Erythema and Edema in vesicles

Determination of the Test Product's SPF Value and PCD:

Calculation of SPF—According to the reference, the mean SPF value (x) is calculated using a minimum of 10 evaluable subjects per formulation. The standard deviation was determined (s). The upper 5% point was obtained from the t distribution table with n−1 degrees of freedom (t). The standard error (SE) was calculated by (s)/Vn (where n equals the number of subjects who provided valid test results).

Therefore, the label SPF value for panels using a minimum of 10 evaluable subjects is the largest whole number less than the mean SPF minus (t*SE).

Label SPF=Mean SPF−(t*SE)

No adverse effects or unexpected reactions of any kind were observed on any of the subjects.

### Evaluation of Sun Protection by SPF Determination (FDA)—Water Resistant—80 Minute Water Immersion

### AMALabNo.: M-7372

| | | | | | | | STD | | | |
|---|---|---|---|---|---|---|---|---|---|---|
| Subject | | MED/ | I | Skin | MED I | MED II | (7% PadO/ | WR | SPF Values | |
| ID# | Sex | Hr | (Amps) | Type | J/M² | J/M² | 3% Oxyb) | Control | Static | WR |
| 46 8676 | F | 125.5 | 6.0 | II | 46.20 | 46.20 | 18.75 | 15.00 | 57.50 | 57.50 |
| 68 4953 | F | 127.4 | 7.1 | I | 30.33 | 30.33 | 16.30 | 18.00 | 66.00 | 66.00 |
| 58 7412 | F | 126.8 | 6.0 | II | 46.20 | 46.20 | 18.75 | 18.00 | 66.00 | 66.00 |
| 03 0493 | F | 127.6 | 6.0 | II | 30.33 | 30.33 | 16.30 | 15.00 | 57.50 | 57.50 |
| 72 8719 | F | 127.3 | 6.3 | II | 30.33 | 30.33 | 16.30 | 15.00 | 57.50 | 57.50 |
| 56 2086 | F | 126.8 | 6.0 | I | 30.33 | 30.33 | 18.75 | 15.00 | 66.00 | 57.50 |
| 52 1152 | F | 125.7 | 6.0 | II | 30.33 | 30.33 | 16.30 | 15.00 | 57.50 | 57.50 |
| 58 8637 | F | 125.4 | 6.0 | II | 46.20 | 46.20 | 18.75 | 18.00 | 66.00 | 57.50 |
| 58 3948 | M | 127.2 | 5.6 | II | 46.20 | 46.20 | 16.30 | 15.00 | 66.00 | 50.00 |
| 63 4385 | F | 127.6 | 6.0 | II | 46.20 | 46.20 | 16.30 | 18.00 | 57.50 | 57.50 |

| | | | | |
|---|---|---|---|---|
| MEAN (x) | 17.28 | 16.20 | 61.75 | 58.45 |
| STANDARD DEV (s) | 1.27 | 1.55 | 4.48 | 4.62 |
| STD. ERROR | 0.40 | 0.49 | 1.42 | 1.46 |
| S.E. % OF MEAN | 2.31 | 3.02 | 2.30 | 2.50 |
| N | 10 | 10 | 10 | 10 |
| UPPER 5% t DIST | 2.2622 | 2.2622 | 1.8331 | 1.8331 |
| A VALUES | 0.9051 | 1.1082 | 2.5969 | 2.6755 |
| LABEL SPF | 16 | 15 | 59 | 55 |

MED: Minimal Erythemal; Dose I: Intensity of light source

### CONCLUSIONS

The Sun Protection Factor (SPF) of the above test material (AMA Lab No.: M-7372) when tested on ten subjects as described herein under static and 80 minute water resistant conditions yielded the mean SPF values of 61.75 and 58.45 and the label SPF's of 59 and 55 respectively. The mean SPF of the 7% Padimate 0/3% Oxybenzone standard on the same panel was 17.28 and was within the standard deviation range of the expected SPF of 16.3+/−3.43. The mean water resistant SPF of 15/15 water resistant in house control on the same panel was 16.20.

Thus, the above tested composition according to the present disclosure surprisingly and unexpectedly achieved an SPF of from about 55 to 60 with avobenzone being sufficiently stable to provide this result for at least about 80 minutes.

The term “about,” as used herein, should generally be understood to refer to both numbers in a range of numerals. Moreover, all numerical ranges herein should be understood to include each whole integer, and preferably each number within the range (e.g., 0.9 to 1.2 would include 1 and 1.1, as well).

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those components, amounts, and methods shown and described herein will become apparent to those of ordinary skill in the art and are intended to fall within the scope of the appended claims.

What is claimed is:

1. A sunscreen composition comprising:
a sunscreen component comprising avobenzone in an amount sufficient to provide at least UVA protection;
an alcohol component present in an amount of about 20 weight percent to no more than 65 weight percent of the total sunscreen composition; and
an antioxidant component present in an amount sufficient to impart an antioxidant effect to the sunscreen composition.

2. The sunscreen composition of claim 1, which further comprises a water-penetrating polymer in an amount sufficient to facilitate application of the composition in the presence of water.

3. The sunscreen composition of claim 2, wherein the water-penetrating polymer comprises an alkyl maleate/acrylate copolymer.

4. The sunscreen composition of claim 3, wherein the water-penetrating polymer is present in an amount of about 0.1 to about 5 weight percent of the total composition.

5. The sunscreen composition of claim 1, wherein the sunscreen composition is at least substantially free of oxybenzone, Vitamin A, or both.

6. The sunscreen composition of claim 1, wherein the sunscreen composition is entirely free of oxybenzone and Vitamin A.

7. The sunscreen composition of claim 1, wherein the sufficient amount of sunscreen component comprises 2.2 weight percent to about 7.5 weight percent avobenzone.

8. The sunscreen composition of claim 1, wherein the sufficient amount of sunscreen component comprises about 2.5 weight percent to about 4.5 weight percent avobenzone.

9. The sunscreen composition of claim, wherein the sunscreen component further comprises at least three of: homosalate, octinoxate, octisalate, and octocrylene.

10. The sunscreen composition of claim 1, wherein the alcohol component is present in an amount of about 30 weight percent to about 60 weight percent of the total composition.

11. The sunscreen composition of claim 1, wherein the alcohol component is present in an amount of about 40 weight percent to about 55 weight percent of the total composition.

12. The sunscreen composition of claim 2, wherein the sunscreen composition imparts sufficient water penetration to provide UVA and UVB sunscreen efficacy for at least 80 minutes.

13. The sunscreen composition of claim 1, wherein the sufficient amount of the antioxidant component is from about 0.001 to about 1 weight percent of the total composition.

14. The sunscreen composition of claim 1, wherein the antioxidant component comprises at least one vitamin and at least one organic extract.

15. The sunscreen composition of claim 14, wherein the antioxidant component comprises Vitamin C in oil soluble form, acai extract, and green tea extract.

16. The sunscreen composition of claim 3, wherein the sunscreen component, alcohol component, antioxidant component, and water-penetrating polymer are collectively present in relative amounts to provide a clear sunscreen composition.

17. A sunscreen composition comprising:
a sunscreen component comprising avobenzone, homosalate, octinoxate, octisalate, and octocrylene each present in an amount sufficient to collectively provide UVA and UVB protection;
an alcohol component present in an amount of about 30 weight percent to about 55 weight percent of the total sunscreen composition;
a water-penetrating polymer comprising an alkyl maleate/acrylate copolymer in an amount sufficient to repel water; and
an antioxidant component comprising at least one vitamin present in an amount sufficient to impart an antioxidant effect to the sunscreen composition.

18. A method of preparing a continuous-spray sunscreen composition, which comprises:
providing a sunscreen component comprising avobenzone in an amount sufficient to provide UVA protection;
providing an alcohol component present in an amount of about 20 weight percent up to 65 weight percent of the total sunscreen composition;
providing an antioxidant component present in an amount sufficient to impart an antioxidant effect to the sunscreen composition;
providing a hydrophobic polymer present in an amount sufficient to repel water contacting the composition; and
combining the sunscreen component, the alcohol component, the antioxidant component, and the hydrophobic polymer so as to form a continuous-spray sunscreen.

19. The method of claim 18, wherein the sunscreen composition is selected to be at least substantially free of oxybenzone.

20. The method of claim 18, wherein the sufficient amount of sunscreen component comprises 2.2 weight percent to about 7.5 weight percent avobenzone.

21. The method of claim 18, wherein the alcohol component is provided in an amount of about 30 weight percent to about 60 weight percent of the total composition.

22. The method of claim 18, wherein the antioxidant component comprises at least one vitamin and at least one extract and is provided in an amount of about 0.001 to about 1 weight percent of the total composition.

23. A method for applying a reduced-alcohol sunscreen composition to skin, which comprises:
providing the reduced-alcohol sunscreen composition of claim 2 in a pressurized container adapted to direct a stream of the sunscreen composition in liquid form from the container in a selected direction; and
activating a release mechanism to direct the liquid sunscreen composition in the selected direction onto a person's skin.

24. The method of claim 23, wherein the skin is wet and the sunscreen composition imparts UVA and UVB sunscreen efficacy with an SPF of at least about 20 for at least 80 minutes to the wet skin.

25. The method of claim 23, wherein the sunscreen component, alcohol component, antioxidant component, and hydrophobic polymer are collectively present in relative amounts in the reduced-alcohol sunscreen composition to provide a clear sunscreen composition as applied to the skin.

26. The method of claim 23, wherein the stream comprises a spray or mist, and the release mechanism is triggered by pressure applied to the pressurized container to direct the sunscreen composition on the person's skin.
